(19) United States
(12) Patent Application Publication
(10) Pub. No.: US 2026/0090969 A1
(43) Pub. Date: Apr. 2, 2026

(54) COMPOSITIONS THAT PROTECT CELLS FROM OXIDATIVE AND MITOCHONDRIAL STRESS

(71) Applicant: NUCHIDO LIMITED, Tyne and Wear (GB)

(72) Inventors: Nichola Jane Conlon, Tyne and Wear (GB); Malcolm Philip Young, Tyne and Wear (GB)

(73) Assignee: NUCHIDO LIMITED, Tyne and Wear (GB)

(21) Appl. No.: 19/255,485

(22) Filed: Jun. 30, 2025

Related U.S. Application Data
(63) Continuation of application No. 17/258,258, now abandoned.

(30) Foreign Application Priority Data
Jul. 10, 2018 (GB) 1811312.6

(51) Int. Cl. A61K 8/67; A61K 8/27; A61K 8/49; A61K 31/352; A61K 31/375; A61K 31/455; A61K 33/30; A61Q 19/08

(52) U.S. Cl. CPC A61K 8/675; A61K 8/27; A61K 8/498; A61K 8/676; A61K 31/352; A61K 31/375; A61K 31/455; A61K 33/30; A61Q 19/08; A61K 2800/92

(57) ABSTRACT

There are described compositions comprising an effective amount of a combination of two or more components, said components selected from acacetin, ACTI peptide, alpha-lipolic acid, alprostadil, anisomycin, apigenin, ascorbic acid, astragalus, berberine, β-lapachone, β-hydroxy-beta-methyl-butyrate, Bacopa monnieri, catechin, catechol, chamomile, chrysin, coumestrol, curcumin, dinitrophenol, dinoprost, ellagic acid, (−)-epigallocatechin gallate, green tea extract, fisetin, genistein, ginsenoside RE, glabridin, 18-α-glycyrrhetinic acid, 18-β-glycyrrhetinic acid, glycyrrhizin, hydroquinone, isoquercitrin (EMIQ), kaempferol, kuromanin, leucine, lithium, luteolin, luteolin, luteolinidin, melatonin, menadione, 1-methylnicotinamide (MNA), methyl salicylate, myricetin, nadide, niacin (vitamin B₃), nicotinamide (NAM), nicotinamide mononucleotide (NMN), nicotinamide riboside (NR), nicotinic acid adenine dinucleotide (NaAD), nicotinic acid mononucleotide (NaMN), parsley (Petroselinium crispum), phenylephrine, pokeweed mitogen, 15-Δ prostaglandin J2, puromycin, quercetin, quinolinic acid, retinoic acid, trichostatin A, troxrutin, rutin, tryptophan, vitamin D3, withaferin A, wortmannin and zinc (including salts thereof).

### FIELD OF THE INVENTION

The present invention relates to novel compositions, generally for oral or topical application, for the mitigation of the effects of ageing.

In particular, the present invention relates to compositions and methods of enhancing cell resistance to DNA damage, oxidative stress, mitochondrial dysfunction, or improving DNA repair capacity.

### BACKGROUND TO THE INVENTION

Declining DNA protection and repair are important generators of genomic instability. Genomic instability in turn underlies many diseases whose risk rises exponentially with ageing, such as cancer.

Protecting DNA and other critical cellular machinery from damage, and repairing it if damage occurs, is important to high functioning cells. The protection and repair of such cells is generally associated with youth and health. Furthermore, these protective and repairing functions decline in somatic cells and underlie most of the decline in cell and tissue function with age.

### SUMMARY TO THE INVENTION

We have now found novel compositions comprising certain active components that are suitable for use, inter alia, in enhancing cell resistance to DNA damage, oxidative stress, mitochondrial dysfunction, or improving DNA repair capacity.

Thus, according to a first aspect of the invention there is provided a composition comprising an effective amount of a combination of two or more components, said components selected from acacetin, ACTI peptide, alpha-lipolic acid, alprostadil, anisomycin, apigenin, ascorbic acid, astragalus, berberine, β-lapachone, β-hydroxy-beta-methyl-butyrate, Bacopa monnieri, catechin, catechol, chamomile, chrysin, coumestrol, curcumin, dinitrophenol, dinoprost, ellagic acid, (−)-epigallocatechin gallate, green tea extract, fisetin, genistein, ginsenoside RE, glabridin, 18-α-glycyrrhetinic acid, 18-β-glycyrrhetinic acid, glycyrrhizin, hydroquinone, isoquercitrin (EMIQ), kaempferol, kuromanin, leucine, lithium, luteolin, luteolin, luteolinidin, melatonin, menadione, 1-methylnicotinamide (MNA), methyl salicylate, myricetin, nadide, niacin (vitamin B₃), nicotinamide (NAM), nicotinamide mononucleotide (NMN), nicotinamide riboside (NR), nicotinic acid adenine dinucleotide (NaAD), nicotinic acid mononucleotide (NaMN), parsley (Petroselinium crispum), phenylephrine, pokeweed mitogen, 15-Δ prostaglandin J2, puromycin, quercetin, quinolinic acid, retinoic acid, trichostatin A, troxrutin, rutin, tryptophan, vitamin D3, withaferin A, wortmannin and zinc (including salts thereof); and derivatives thereof; and any combination thereof.

The amount of the active components in the composition according to this aspect of the invention may vary depending upon the nature of the active components, the mode of administration, etc. Exemplary amounts of active components which may be in the composition are from about 1 mg to about 1000 mg; or from about 50 mg to about 900 mg; or from about 100 mg to about 800 mg; or from about 150 mg to about 700 mg; or from about 200 mg to about 600 mg; or from about 250 mg to about 500 mg.

It is desirable to enhance the resistance of cells, especially skin cells, to DNA damage by enhancing the DNA protection and repair capacity of the cells, and by enhancing their ability to withstand oxidative stress and mitochondrial stress and dysfunction.

According to this aspect of the invention there is provided a composition for use in enhancing cell resistance to DNA damage, oxidative stress, mitochondrial dysfunction, or improving DNA repair capacity.

In another aspect of the invention the composition is for use in enhancing cell resistance to DNA damage, e.g. enhancing skin cell resistance to DNA damage.

In another aspect of the invention the composition is for use in enhancing cell resistance to oxidative stress, e.g. enhancing skin cell resistance to oxidative stress.

In another aspect of the invention the composition is for use in enhancing cell resistance to mitochondrial dysfunction, e.g. enhancing cell resistance to mitochondrial dysfunction.

In another aspect of the invention the composition is for use in improving a cell's DNA repair capacity, e.g. improving a skin cell's DNA repair capacity.

In one aspect of the invention the composition is for use in enhancing cell resistance to DNA damage, oxidative stress, mitochondrial dysfunction, and improving DNA repair capacity.

Thus, according to this aspect of the invention there is provided a composition comprising an effective amount of one or more components, said components selected from acacetin, ACTI peptide, alpha-lipolic acid, alprostadil, anisomycin, apigenin, ascorbic acid, astragalus, berberine, β-lapachone, β-hydroxy-beta-methyl-butyrate, Bacopa monnieri, catechin, catechol, chamomile, chrysin, coumestrol, curcumin, dinitrophenol, dinoprost, ellagic acid, (−)-epigallocatechin gallate, green tea extract, fisetin, genistein, ginsenoside RE, glabridin, 18-α-glycyrrhetinic acid, 18-β-glycyrrhetinic acid, glycyrrhizin, hydroquinone, isoquercitrin (EMIQ), kaempferol, kuromanin, leucine, lithium, luteolin, luteolin, luteolinidin, melatonin, menadione, 1-methylnicotinamide (MNA), methyl salicylate, myricetin, nadide, niacin (vitamin B₃), nicotinamide (NAM), nicotinamide mononucleotide (NMN), nicotinamide riboside (NR), nicotinic acid adenine dinucleotide (NaAD), nicotinic acid mononucleotide (NaMN), parsley (Petroselinium crispum), phenylephrine, pokeweed mitogen, 15-4 prostaglandin J2, puromycin, quercetin, quinolinic acid, retinoic acid, trichostatin A, troxrutin, rutin, tryptophan, vitamin D3, withaferin A, wortmannin and zinc (including salts thereof); and derivatives thereof; and any combination thereof; for use in enhancing cell resistance to DNA damage, oxidative stress, mitochondrial dysfunction, or improving DNA repair capacity.

Thus, according to a further aspect of the invention there is provided a composition comprising an effective amount of one or more components, said components selected from acacetin, ACTI peptide, alpha-lipolic acid, alprostadil, anisomycin, apigenin, ascorbic acid, astragalus, berberine, β-lapachone, β-hydroxy-beta-methyl-butyrate, Bacopa monnieri, catechin, catechol, chamomile, chrysin, coumestrol, curcumin, dinitrophenol, dinoprost, ellagic acid, (−)-epigallocatechin gallate, green tea extract, fisetin, genistein, ginsenoside RE, glabridin, 18-α-glycyrrhetinic acid, 18-β-glycyrrhetinic acid, glycyrrhizin, hydroquinone, isoquercitrin (EMIQ), kaempferol, kuromanin, leucine, lithium, luteolin, luteolin, luteolinidin, melatonin, menadione, 1-methylnicotinamide (MNA), methyl salicylate, myricetin, nadide, niacin (vitamin B₃), nicotinamide (NAM), nicotinamide mononucleotide (NMN), nicotinamide riboside (NR), nicotinic acid adenine dinucleotide (NaAD), nicotinic acid mononucleotide (NaMN), parsley (Petroselinium crispum), phenylephrine, pokeweed mitogen, 15-4 prostaglandin J2, puromycin, quercetin, quinolinic acid, retinoic acid, trichostatin A, troxrutin, rutin, tryptophan, vitamin D3, withaferin A, wortmannin and zinc (including salts thereof); and derivatives thereof; and any combination thereof; for use in enhancing a cell's DNA repair capacity.

According to this aspect of the invention there is provided a composition as herein described for use in enhancing cell resistance to DNA damage, oxidative stress, mitochondrial dysfunction, or improving DNA repair capacity.

The amount of the active component in the composition according to this aspect of the invention may vary depending upon the nature of the active component, the mode of administration, etc. Exemplary amounts of active component which may be in the composition are from about 1 mg to about 1000 mg per day; or from about 50 mg to about 900 mg; or from about 100 mg to about 800 mg; or from about 150 mg to about 700 mg; or from about 200 mg to about 600 mg; or from about 250 mg to about 500 mg.

According to a further aspect of the invention there is provided a composition as herein described for use in enhancing cell resistance, e.g. skin cell resistance, to DNA damage, oxidative stress, mitochondrial dysfunction, or improving DNA repair capacity comprising topically applying an effective amount of the composition to an individual's skin.

According to a further aspect of the invention there is provided a composition as herein described for use in the mitigation, alleviation or improvement of the effects of ageing by enhancing cell resistance, e.g. skin cell resistance, to DNA damage, oxidative stress, mitochondrial dysfunction, or improving DNA repair capacity.

According to this aspect of the invention there is provided a composition as herein described for use in the mitigation, alleviation or improvement of the effects of ageing as herein described.

The effects of ageing may include age related skin conditions, skin conditions related to sun exposure, skin conditions related to pollution exposure, skin conditions related to oxidative stress, and skin conditions related to lifestyle choices, such as diet, alcohol and/or smoking. In addition, the compositions of the invention may be advantageous in the mitigation, alleviation or improvement of skin conditions related to inflammatory skin disorders and skin conditions related autoimmune disease skin disorders. The compositions of the invention may be advantageous in the mitigation, alleviation or improvement of other age related conditions, such as, but not limited to, increased frailty, loss of resilience, loss of muscle strength, loss of muscle endurance, loss of energy, loss of cognitive sharpness, loss of memory, etc. More specifically, the compositions of the invention may be advantageous in the mitigation, alleviation or improvement of other age related conditions, such as, but not limited to, atherosclerosis and cardiovascular disease, cancer, arthritis, cataracts, osteoporosis, type 2 diabetes, hypertension and Alzheimer's disease; the incidence of which increases with aging.

Age related skin conditions that may be mitigated, alleviated or improved, shall include, but shall not be limited to, one or more of sagging, wrinkles, skin elasticity, skin ageing, skin moisture, wounds, acne, skin darkening, skin whitening, pigmentation, age-spots, loss of radiance, puffiness, uneven skin tone, redness, rosacea, loss of barrier function, loss of skin resilience, loss of firmness, stretch-marks, cellulite and dryness.

Skin conditions related to sun exposure that may be mitigated, alleviated or improved, include, but shall not be limited to, one or more of actinic keratoses, freckles, lentigines or age spots, moles, photosensitivity, polymorphous light eruption, seborrheic keratoses, skin cancer (such as melanoma, squamous cell carcinoma, basal cell carcinoma), solar elastosis or wrinkles and sun burn.

Skin conditions related to inflammatory skin disorders that may be mitigated, alleviated or improved, include, but shall not limited to, one or more of acne, asteatotic eczema, atopic dermatitis, contact dermatitis, discoid eczema, eczematous drug eruptions, erythema multiforme, erythroderma, gravitational/varicose eczema, hand eczema, keratosis lichenoides chronica, lichen nitidus, lichen planus, lichen simplex, lichen striatus, mycosis fungoides, pityriasis lichenoides, psoriasis, seborrheic dermatitis, Stevens-Johnson Syndrome, toxic epidermal necrolysis and vasculitis.

Skin conditions related autoimmune disease skin disorders that may be mitigated, alleviated or improved, include, but shall not limited to, one or more of alopecia areata, bullous pemphigoid, dermatomyositis, dystrophic epidermolysis bullosa, eosinophilic fasciitis, pemphigus vulgaris, psoriasis, pyoderma gangrenosum, scleroderma, systemic lupus erythematosus and vitiligo.

According to further aspect of the invention there is provided a composition as herein described for use in the mitigation, alleviation or improvement of the effects of an autoimmune disorder by improving a cell's resistance, e.g. skin cell resistance, to DNA damage and/or enhancing the skin cell's DNA repair capacity.

Such autoimmune disorders and related immune disorders shall include, but shall not be limited to, systemic lupus erythematosus (SLE), rheumatoid arthritis, non-glomerular nephrosis, psoriasis, chronic active hepatitis, ulcerative colitis, Crohn's disease, Behcet's disease, chronic glomerulonephritis, chronic thrombocytopeni purpura, and autoimmune haemolytic anaemia.

The composition of the present invention may be administered topically, orally or parenterally; or may comprise controlled, modified or extended release formulations comprising suitable mitigation amounts of the desired active components in the form of powders, granules, sterile parenteral solutions or suspensions, oral solutions or suspensions, oil water emulsions as well as implants and microencapsulated delivery systems.

### Parenteral Administration

Thus, according to one aspect of the invention there is provided the composition as herein described for parenteral administration.

When the composition of the invention is administered parenterally, it may be in the form of an intramuscular, intravenous, subcutaneous, intraperitoneal, local or transdermal injections.

### Topical Administration

Preferably, the composition of the invention may be administered topically or transdermally. Thus, according to this aspect of the invention there is provided the composition as herein described for topical administration. According to a further aspect of the invention there is provided the composition as herein described for transdermal administration.

Suitable formulations for topical or transdermal application include an effective amount of the composition of the invention comprise the active components as herein defined with one or more carriers. Carriers include absorbable pharmacologically acceptable solvents to assist passage into the skin of the host.

Suitable formulations for topical application, e.g., to the skin and eyes, include aqueous solutions, suspensions, ointments, creams, gels, sprayable formulations, transdermal patch or bandage e.g., for delivery by aerosol or the like. Such topical delivery systems will in particular be appropriate for dermal application, for prophylactic use in sun creams, lotions, sprays and the like. They are thus particularly suited for use in topical, including cosmetic, formulations well known in the art. Such formulations may contain solubilisers, stabilizers, tonicity enhancing agents, buffers and preservatives.

Transdermal devices may be in the form of a bandage comprising a backing member, a reservoir containing the composition of the invention optionally with carriers, optionally a rate controlling barrier to deliver the composition of the invention to the skin of the host at a controlled and predetermined rate over a prolonged period of time, and means to secure the device to the skin.

Compositions of this invention may also include a cosmetically acceptable carrier. Amounts of the carrier may range from 1 to 99.9%, preferably from 70 to 95%, optimally from 80 to 90%. Among the useful carriers are water, emollients, fatty acids, fatty alcohols, thickeners and combinations thereof. The carrier may be aqueous, anhydrous or an emulsion. Preferably, the compositions are aqueous, especially water and oil emulsions of the water-in-oil or oil-in-water type or multiple emulsions of the water-in-oil-in-water or oil-in-water-in-oil variety. Water when present may be in amounts ranging from 5 to 95%, preferably from about 20 to about 70%, optimally from 35 to 60% by weight.

Emollient materials may serve as cosmetically acceptable carriers. These may be in the form of silicone oils, natural or synthetic esters, hydrocarbons, alcohols and fatty acids. Amounts of the emollients may range anywhere from 0.1 to 95%, preferably between 1 and 50% by weight of the composition.

Silicone oils may be divided into the volatile and non-volatile variety. The term “volatile” as used herein refers to those materials which have a measurable vapour pressure at ambient temperature. Volatile silicone oils are preferably chosen from cyclic (cyclomethicone) or linear polydimethylsiloxanes containing from 3 to 9, preferably from 5 to 6, silicon atoms. Non-volatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from 5×10-6 to 0.1 m²/sat 25° C.

Among the preferred non-volatile emollients useful in the present compositions are the polydimethyl siloxanes having viscosities from 1×10<−5> to about 4×10<−4>m<2>/sat 25° C. Another class of non-volatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester emollients are:
a) Alkyl esters of saturated fatty acids having 10 to 24 carbon atoms. Examples thereof include behenyl neopentanoate, isononyl isonanonoate, isopropyl myristate and octyl stearate.
b) Ether-esters such as fatty acid esters of ethoxylated saturated fatty alcohols.
c) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C1-C30 alcohols.
d) Wax esters such as beeswax, spermaceti wax and tribehenin wax.
e) Sugar ester of fatty acids such as sucrose polybehenate and sucrose polycottonseedate.

Natural ester emollients principally are based upon mono-, di- and tri-glycerides. Representative glycerides include sunflower seed oil, cottonseed oil, borage oil, borage seed oil, primrose oil, castor and hydrogenated castor oils, rice bran oil, soybean oil, olive oil, safflower oil, shea butter, jojoba oil and combinations thereof. Animal derived emollients are represented by lanolin oil and lanolin derivatives. Amounts of the natural esters may range from 0.1 to 20% by weight of the compositions.

Hydrocarbons which are suitable cosmetically acceptable carriers include petrolatum, mineral oil, C11-C13 isoparaffins, polybutenes and especially isohexadecane, available commercially as Permethyl 101A from Presperse Inc.

Fatty acids having from 10 to 30 carbon atoms may also be suitable as cosmetically acceptable carriers. Illustrative of this category are pelargonic, lauric, myristic, palmitic, stearic, isostearic, oleic, linoleic, linolenic, hydroxystearic and behenic acids and mixtures thereof.

Fatty alcohols having from 10 to 30 carbon atoms are another useful category of cosmetically acceptable carrier. Illustrative of this category are stearyl alcohol, lauryl alcohol, myristyl alcohol, oleyl alcohol and cetyl alcohol and mixtures thereof.

Thickeners or rheology modifiers can be utilized as part of the cosmetically acceptable carrier of compositions according to the present invention. Typical thickeners include crosslinked acrylates (e.g. Carbopol®), hydrophobically modified acrylates (e.g. Carbopol®), polyacrylamides (e.g. Sepigel®), acryloylmethylpropane sulfonic acid/salt polymers and copolymers (e.g. Aristoflex® and AVO®), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Inorganics may also be utilized as thickeners, particularly clays such as bentonites and hectorites, fumed silicas, talc, calcium carbonate and silicates such as magnesium aluminium silicate (Veegum®). Amounts of the thickener may range from 0.0001 to 10%, usually from 0.001 to 1%, or from 0.01 to 0.5%.

Emollients that can be used, especially for products intended to be applied to the face, to improve sensory properties and are chosen from the group of polypropylene glycol-14 butyl ether otherwise known as Tegosoft PBE, or PPG15 stearyl ether such as Tegosoft E, other oils such as esters, specifically, isopropyl myristate, isopropyl palmitate, other oils could include castor oils and derivatives thereof.

Humectants of the polyhydric alcohol-type can be employed as cosmetically acceptable carriers. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1, 2, 6-hexanetriol, ethoxylated glycerol, propoxylated glycerol and mixtures thereof. The amount of humectant may range anywhere from 0.5 to 50%, preferably between 1 and 15% by weight of the composition.

Skin moisturizers, e.g. hyaluronic acid and/or its precursor N-acetyl glucosamine may be included. N-acetyl glucosamine may be found in shark cartilage or shitake mushrooms and are available commercially from Maypro Industries, Inc. (New York). Other moisturizing agents include hydroxypropyl tri (C1-C3 alkyl) ammonium salts. These salts may be obtained in a variety of synthetic procedures, most particularly by hydrolysis of chlorohydroxypropyl tri (C1-C3 alkyl) ammonium salts. A particular species is 1, 2-dihydroxypropyltrimonium chloride, wherein the C1-C3 alkyl is a methyl group. Amounts of the salt may range from 0.2 to 30%, and preferably from 0.5 to 20%, optimally from 1% to 12% by weight, of the topical composition, including all ranges subsumed therein.

Ordinarily the C1-C3 alkyl constituent on the quaternized ammonium group will be methyl, ethyl, n-propyl, isopropyl or hydroxyethyl and mixtures thereof. Particularly preferred is a trimethyl ammonium group known through INCI nomenclature as a “trimonium” group. Any anion can be used in the quat salt. The anion may be organic or inorganic with proviso that the material is cosmetically acceptable. Typical inorganic anions are halides, sulfates, phosphates, nitrates and borates. Most preferred are the halides, especially chloride. Organic anionic counter ions include methylsulfate, toluoyl sulfate, acetate, citrate, tartrate, lactate, gluconate, and benzenesulfonate.

Still other moisturizing agents which may be used, especially in conjunction with the aforementioned ammonium salts include substituted urea like hydroxymethyl urea, hydroxyethyl urea, hydroxypropyl urea; bis-(hydroxymethyl) urea; bis(hydroxyethyl) urea; bis(hydroxypropyl) urea; N,N′-dihydroxymethyl urea; N,N′-di-hydroxyethyl urea; N,N′-di-hydroxypropyl urea; N, N,N′-tri-hydroxyethyl urea; tetra (hydroxymethyl) urea; tetra (hydroxyethyl) urea; tetra (hydroxypropyl urea; N-methyl, N′-hydroxyethyl urea; N-ethyl-N′-hydroxyethyl urea; N-hydroxypropyl-N′-hydroxyethyl urea and N,N′-dimethyl-N-hydroxyethyl urea. Where the term hydroxypropyl appears, the meaning is generic for either 3-hydroxy-n-propyl, 2-hydroxy-n-propyl, 3-hydroxy-i-propyl or 2-hydroxy-i-propyl radicals. Most preferred is hydroxyethyl urea. The latter is available as a 50% aqueous liquid. Amounts of substituted urea that may be used in the topical composition of this invention range from 0.01 to 20%, or from 0.5 to 15%, or from 2 to 10%.

When ammonium salt and substituted urea are used, in a most especially preferred embodiment at least from 0.01 to 25%, or from 0.2 to 20%, or from 1 to 15% humectant, like glycerine, is used. Further moisturizing agents for use herein include petrolatum and/or various aquaporin manipulating actives and/or oat kernel flour.

In one embodiment, the pH of the topical composition is between 3.5 and 8.5. In some embodiments, the pH of the topical composition is between pH 3.5 and pH 8. In some embodiments, the pH of the topical composition is between pH 5 to pH 7.8. In some embodiments, the pH of the topical composition is between 5 and 7.5.

In some embodiments, the topical composition of the present invention contains sunscreen. These are typically a combination of organic and inorganic sunscreens. It may be particularly desirable to include both UV-A and UV-B radiation sunscreens.

UV-B sunscreen oil may be selected from the class of cinnamic acid, salicylic acid, diphenyl acrylic acid, or derivatives thereof. The UV-B sunscreen oil may include one or more of octyl salicylate, 3, 3, 5-trimethylcyclohexyl 2-hydroxybenzoate, ethylhexyl salicylate, 2-ethylhexyl 2-cyano-3, 3-diphenyl-2-propenoate, or 2-ethylhexyl-4-methoxycinnamate (also known as octyl methoxycinnamate or “OMC”). Such UV-B sunscreen oils are typically commercially available, such as Octisalate™ (octyl salicylate), Homosalate™ (3, 3, 5-trimethyleyclohexyl 2-hydroxybenzoate), NeoHeliopan™ (a range of organic UV filters including OMC (Neo Heliopan AV™) and ethylhexyl salicylate (Neo Heliopan OS™), Octocrylene™ and Milestab 3039™ (2-ethylhexyl-2-cyano-3, 3-diphenyl-2-propenoate) or Parsol MCX™ (2-ethylhexyl-4-methoxycinnamate). The amount of UV-B sunscreen oil in the topical composition may be 0.1 wt % to 20 wt %, or 0.2 wt % to 10 wt %, or 0.5 wt % to 7 wt %, or 2 wt % to 6 wt %.

The topical composition may further include a UV-B sunscreen that is water-soluble. The water soluble UV-B sunscreen may also include phenylbenzimidazole sulfonic acid (also known as ensulizole), 4-aminobenzoic acid (also known as para-aminobenzoic acid or “PABA”), or both.

The topical composition of any one of the above embodiments may further include 0.1 wt % to 10 wt % of a UV-A sunscreen oil. The UV-A sunscreen oil may include one or more of 4-t-butyl-4′-methoxydibenzoylmethane (“avobenzone”), 2-methyldibenzoylmethane, 4-methyl-dibenzoyl-ethane, 4-isopropyldibenzoyl-methane, 4-tert-butyldibenzoylmethane, 2,4-dimethyldibenzoylmethane, 2, 5-dimethyldibenzoylmethane, 4,4′-diisopropyldibenzoylmethane, 2-methyl-5-isopropyl-4′-methoxy-dibenzoylmethane, 2-methyl-5-tert-butyl-4′-methoxy-dibenzoylmethane, 2,4-dimethyl-4′-methoxydibenzoylmethane, 2, 6-dimethyl-4-tert-butyl-4′methoxy-dibenzoylmethane, diethylaminohydroxybenzoyl hexyl benzoate, ecamsule, or methyl anthranilate. The amount of UV-A sunscreen oil in the topical composition may be 0.5 wt % to 7 wt %, or 1 wt % to 5 wt %.

Additional suitable sunscreen oils suitable for use in the topical composition include those commercially available from BASF corporation: Uvinul T-150 (Ethylhexyl triazone; a UV-B sunscreen oil), Uvinul A Plus (Diethylamino hydroxybenzoyl hexyl benzoate; a UV-A sunscreen oil), Tinosorb S (bis-ethylhexyloxyphenol methoxyphenyl triazine; a UV-A and UV-B sunscreen oil), Tinosorb M (methylene bisbenzotriazolyl tetramethylbutylphenol; a UV-A and UV-B sunscreen oil). Bisdisulizone disodium may also be included in the topical composition.

A particular combination of UV-A and UV-B sunscreen oils is avobenzone and 2-ethylhexyl-4-methoxycinnamate.

In some embodiments, the sunscreen is an inorganic sunscreen. Examples of inorganic sunscreens suitable for use in the skin care composition of the present invention include, but are not limited to, microfine titanium dioxide, zinc oxide, polyethylene and various other polymers. By the term “microfine” is meant particles of average size ranging from 10 to 200 nm, alternatively from 20 to 100 nm. Amounts of the sunscreen when present in a skin care formulation according to some embodiments of the present invention may range from 0.1% to 30%, alternatively from 2% to 20%, alternatively from 4% to 10%.

The inventive composition may include a skin lightening compound, in addition to nicotinamide included herein, to obtain optimum skin lightening performance at an optimum cost. Illustrative substances are placental extract, lactic acid, resorcinols (4-substituted, 2,5-disubstituted, 4, 5-disubstituted, and 4, 6 di-substituted, in particular 4-hexyl, 4-methyl, 4-butyl, 4-isopropyl, phenylethyl resorcinols), arbutin, kojic acid, ferulic acid, hydroquinone, resorcinol derivatives including di-substituted resorcinols and combinations thereof. In one embodiment, such skin lightening compound is a tyrosinase inhibitor, most preferably a compound selected from the group consisting of kojic acid, hydroquinone and 4-substituted resorcinols). Also, dicarboxylic acids represented by the formula HOOC—(CxHy)-COOH where x=4 to 20 and y=6 to 40 such as azelaic acid, sebacic acid, oxalic acid, succinic acid, fumaric acid, octadecenedioic acid (e.g. Arlatone DC) or their salts or a mixture thereof, most preferably fumaric acid or salt thereof, especially di-sodium salt. It has been found that combination with 12HSA with fumaric acid or salts thereof are particularly preferred, especially for skin lightening formulations. Amounts of these agents may range from 0.1 to 10%, preferably from 0.5 to 2% by weight of the composition. It is preferred that the skin lightening coactive according to the invention is 4-alkyl resorcinol and/or 12-hydroxy stearic acid.

Another preferred ingredient of the inventive compositions is a retinoid. As used herein, “retinoid” includes all natural and/or synthetic analogues of Vitamin A or retinol-like compounds which possess the biological activity of Vitamin A in the skin as well as the geometric isomers and stereoisomers of these compounds. The retinoid is preferably retinol, retinol esters (e.g., C2-C22 alkyl esters of retinol, including retinyl palmitate, retinyl acetate, retinyl propionate), retinal, and/or retinoic acid (including all-trans retinoic acid and/or 13-cis-retinoic acid), more preferably retinoids other than retinoic acid. These compounds are well known in the art and are commercially available from a number of sources, e.g., Sigma Chemical Company (St. Louis, Mo.), and Boerhinger Mannheim (Indianapolis, Ind.). Other retinoids which are useful herein are described in U.S. Pat. Nos. 4,677,120; 4,885,311; 5,049,584; 5,124,356. Other suitable retinoids are tocopheryl-retinoate [tocopherol ester of retinoic acid (trans- or cis-), adapalene {6-[3-(1-adamantyl)-4-methoxyphenyl]-2-naphthoic acid}, and tazarotene (ethyl 6-[2-(4, 4-dimethylthiochroman-6-yl)-ethynyl] nicotinate). Preferred retinoids are retinol, retinyl palmitate, retinyl acetate, retinyl propionate, retinal and combinations thereof. The retinoid is preferably substantially pure, more preferably essentially pure. The compositions of this invention may contain a safe and effective amount of the retinoid, such that the resultant composition is safe and effective for regulating keratinous tissue condition, preferably for regulating visible and/or tactile discontinuities in skin, more preferably for regulating signs of skin aging, even more preferably for regulating visible and/or tactile discontinuities in skin texture associated with skin aging. The compositions preferably contain from 0.005% to 2%, or from 0.01% to 2%, retinoid. Retinol is preferably used in an amount of 0.01% to 0.15%; retinol esters are preferably used in an amount of from 0.01% to 2% (e.g., 1%); retinoic acids are preferably used in an amount of 0.01% to 0.25%; tocopheryl-retinoate, adapalene, and tazarotene are preferably used in an amount of from 0.01% to 2%.

A variety of herbal extracts may optionally be included in compositions of this invention. Illustrative are pomegranate, white birch (Betula Alba), green tea, chamomile, liquorice and extract combinations thereof. The extracts may either be water soluble or water-insoluble carried in a solvent which respectively is hydrophilic or hydrophobic. Water and ethanol are the preferred extract solvents.

The topical composition may further include about 0.1 wt % to about 8 wt % of a film forming polymer. Such film-forming polymers include, but are not limited to, polyalkyleneoxy terminated polyamides (e.g., INCI name: Polyamide-3, Polyamide-4), polyether polyamides (e.g., INCI name: Polyamide-6), mixed acid terminated polyamides (e.g., INCI name: Polyamide-7), and ester terminated poly (ester-amides) (e.g., INCI name: Polyamide-8). Such film forming polymers may be synthesized or are available commercially, such as under the Sylvaclear™ line of products by Arizona Chemical Company, LLC and the OleoCraft™ line of products by Croda International PLC. Film-forming polymers also include, but are not limited to, the INCI named Polyester-5 (e.g., Eastman AQ™ 38S Polymer), PPG-17/IPDI/DMPA Copolymer (e.g., Avalure™ UR 450 Polymer), Acrylates Copolymer (e.g., Avalure™ AC 120 Polymer), and polysaccharides such as Xilogel (tamarin gum), lotus bean gums, tara gum, beta glucan, pullulan, carboxymethyl cellulose, hydroxypropyl cellulose, sodium alginate, potato starch, carrageenan. The film forming polymer may include combinations of any two or more of the polymers recited above. The amount of film forming polymer in the topical composition may be 0.1 wt % to 8 wt %.

Preservatives can desirably be incorporated into the compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preservatives are iodopropynyl butyl carbamate, phenoxyethanol, caprylyl glycol, C1-6 parabens (especially, methyl paraben and/or propyl paraben), imidazolidinyl urea, sodium dehydroacetate and benzyl alcohol. The preservatives should be selected having regard for the use of the composition and possible incompatibilities between the preservatives and other ingredients in the emulsion. Preservatives may be employed in amounts ranging from 0.01% to 2%. One particular combination is octocrylene and caprylyl glycol, since caprylyl glycol has been disclosed to enhance UVA and UVB protection.

Anti-fungal agents suitable for inclusion in topical compositions are well known to one of skill in the art. Examples include, but are not limited to, climbazole, ketoconazole, fluconazole, clotrimazole, miconazole, econazole, etaconazole, terbinafine, salts of any one or more of these (e.g., hydrochloride salts), zinc pyrithione, selenium disulfide, and combinations of any two or more thereof.

In some embodiments, the topical compositions of the present invention include vitamins. Illustrative vitamins are Vitamin A (retinol), Vitamin B2, Vitamin B3 (niacin), Vitamin B6, Vitamin B12, Vitamin C, Vitamin D, Vitamin E, Vitamin K and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. In some embodiments, the Vitamin B3 derivative is nicotinamide riboside. In some embodiments, the Vitamin B6 derivative is pyridoxine palmitate. Flavonoids may also be useful, particularly glucosyl hesperidin, rutin, and soy isoflavones (including genistein, daidzein, equol, and their glucosyl derivatives) and mixtures thereof. Total amount of vitamins or flavonoids when present may range from 0.0001% to 10%, alternatively from 0.001% to 10%, alternatively from 0.01% to 10%, alternatively from 0.1% to 10%, alternatively from 1% to 10%, alternatively from 0.01% to 1%, alternatively from 0.1% to 0.5%.

In some embodiments, the topical compositions of the present invention include an enzyme such as, for example oxidases, proteases, lipases and combinations thereof. In some embodiments, the topical compositions of the present invention includes superoxide dismutase, commercially available as Biocell SOho aD from the Brooks Company, USA.

In some embodiments, the topical compositions of the present invention include desquamation promoters. In some embodiments, the topical compositions of the present invention include desquamation promoters at a concentration from 0.01% to 15%, alternatively from 0.05% to 15% alternatively from 0.1% to 15%, alternatively from 0.5% to 15%.

Illustrative desquamation promoters include monocarboxylic acids. Monocarboxylic acids may be substituted or unsubstituted with a carbon chain length of up to 16. In some embodiments, the carboxylic acids are the alpha-hydroxycarboxylic acids, beta-hydroxycarboxylic or polyhydroxycarboxylic acids. The term “acid” is meant to include not only the free acid but also salts and C1-C30 alkyl or aryl esters thereof and lactones generated from removal of water to form cyclic or linear lactone structures. Representative acids include glycolic, lactic malic and tartaric acids. In some embodiments, the salt is ammonium lactate. In some embodiments, the beta-hydroxycarboxylic acid is salicylic acid. In some embodiments, the phenolic acids include ferulic acid, salicylic acid, kojic acid and their salts.

In some embodiments, the at least one additional component may be present from 0.000001% to 10%, alternatively from 0.00001% to 10%, alternatively from 0.0001% to 10%, alternatively from 0.001% to 10%, alternatively from 0.01% to 10%, alternatively from 0.1% to 10%, alternatively from 0.0001% to 1% by weight of the composition. Colorants, opacifiers or abrasives may also be included in compositions of the present invention. The colorants, opacifiers or abrasives may be included at a concentration from 0.05% to 5%, alternatively between 0.1% and 3% by weight of the composition.

In some embodiments, the personal care product of the present invention may also include a peptide, such as, for example, the commercially available pentapeptide derivative-Matrixyl™, which is commercially available from Sederma, France. In another example, in some embodiments, the personal care product of the present invention may also include Carnosine.

The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the topical cosmetic skin care industry, which are suitable for use in the compositions of the present invention. Examples include: antioxidants, binders, biological additives, buffering agents, colorants, thickeners, polymers, astringents, fragrance, humectants, opacifying agents, conditioners, exfoliating agents, pH adjusters, preservatives, natural extracts, essential oils, skin sensates, skin soothing agents, and skin healing agents.

The topical compositions of the present invention are preferably non-solid. The compositions of the invention are preferably leave-on compositions. The compositions of the present invention are preferably leave-on compositions to be applied to remain on the skin. These leave-on compositions are to be distinguished from compositions which are applied to the skin and subsequently removed by either washing, rinsing, wiping, or the like either after or during the application of the product. Surfactants typically used for rinse-off compositions have physico-chemical properties giving them the ability to generate foam/lather in-use with ease of rinse; they can consist of mixtures of anionic, cationic, amphoteric, and non-ionic. Surfactants used in leave-on compositions on the other hand are not required to have such properties. Rather, as leave-on compositions are not intended to be rinsed-off they need to be non-irritating and therefore it is necessary to minimize the total level of surfactant and the total level of anionic surfactant in leave-on compositions. The total level of surfactant in the inventive compositions is preferably from 1% to no more than 10%, more preferably below 8%, most preferably at most 5%, optimally at most 3%.

In some embodiments, anionic surfactants are present in the leave-on skin care composition in an amount of 0.01% to at most 5% by weight of the composition, alternatively from 0.01% to 4% by weight of the composition, alternatively from 0.01% to 3% by weight of the composition, alternatively from 0.01% to 2% by weight of the composition, alternatively substantially absent (less than 1%, or less than 0.1%, or less than 0.01%). In some embodiments, the total level of surfactant in the skin care compositions is no more than 10%, alternatively below 8%, alternatively at most 5%.

In some embodiments, the surfactant is selected from the group consisting of anionic, non-ionic, cationic and amphoteric actives.

In some embodiments, non-ionic surfactants are those with a C10-C20 fatty alcohol or acid hydrophobe condensed with from 2 to 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan, mono- and di-C8-C20 fatty acids; and polyoxyethylene sorbitan as well as combinations thereof. In some embodiments, the non-ionic surfactant is selected from the group consisting of alkyl polyglycosides, saccharide fatty amides (e.g. methyl gluconamides) and trialkylamine oxides.

Amphoteric surfactants suitable in skin care compositions according to some embodiments of the present invention include cocoamidopropyl betaine, C12-C20 trialkyl betaines, sodium lauroamphoacetate, and sodium laurodiamphoacetate.

Anionic surfactants suitable in skin care compositions according to some embodiments of the present invention include soap, alkyl ether sulfates and sulfonates, alkyl sulfates and sulfonates, alkylbenzene sulfonates, alkyl and dialkyl sulfosuccinates, C8-C20 acyl isethionates, C8-C20 alkyl ether phosphates, C8-C20 sarcosinates, C8-C20 acyl lactylates, sulfoacetates and combinations thereof.

The compositions of the present invention are typically in the form of emulsions, which may be oil-in-water, or water-in-oil. In some embodiments the topical compositions are vanishing creams and creams or lotions based on an oil-in-water emulsion. Vanishing cream base is one which comprises 5 to 40% fatty acid and 0.1 to 20% soap. In such creams, the fatty acid is preferably substantially a mixture of stearic acid and palmitic acid and the soap is preferably the potassium salt of the fatty acid mixture, although other counter ions and mixtures thereof can be used. The fatty acid in vanishing cream base is often prepared using hysteric acid which is substantially (generally about 90 to 95%) a mixture of stearic acid and palmitic acid. A typical hysteric acid comprises about 52-55% palmitic acid and 45-48% stearic acid of the total palmitic-stearic mixture. Thus, inclusion of hysteric acid and its soap to prepare the vanishing cream base is within the scope of the present invention. It is particularly preferred that the composition comprises higher than 7%, preferably higher than 10%, more preferably higher than 12% fatty acid. A typical vanishing cream base is structured by a crystalline network and is sensitive to the addition of various ingredients.

In one embodiment, the topical composition is formulated as a water-in-oil emulsion with cystine substantially solubilized in the aqueous phase. In one embodiment, the topical composition is formulated as a water-in-oil emulsion with cystine in the aqueous droplets, with at least 90% of the droplets having a diameter in the range of from 100 nm to 20 microns, or in the alternative from 200 nm to 20 microns, or to 10 microns.

In some embodiments, the topical composition is formulated as a facial mask. In some embodiments, the topical composition is formulated as a facial mask according to the formulations described in U.S. Pat. No. 5,139,771. In some embodiments, the topical composition is formulated as a facial mask according to the formulations described in U.S. Pat. No. 4,933,177. In some embodiments, the topical composition is formulated as a facial mask according to the formulations described in U.S. Pat. No. 6,001,367.

### Oral Administration

Thus, according to one aspect of the invention there is provided the composition as herein described for oral administration.

When the composition of the invention is administered orally, it may be in the form of tablets or capsules.

The compositions of the invention can be made up in a solid form including capsules, tablets, pills, granules, powders, food bar or confectionery; or in a liquid form including solutions, suspensions or emulsions or in the form of a syrup, linctus, elixir, a liquid beverage, such as a yoghurt drink, or a foodstuff, such as a yoghurt.

The compositions can be subjected to conventional operations such as sterilization and/or can contain conventional inert diluents, lubricating agents, or buffering agents, as well as adjuvants, such as preservatives, stabilizers, wetting agents, emulsifiers and buffers etc.

Typically, when the compositions are in the form of tablets or capsules, e.g. gelatin capsules, the compositions may comprise the active component or components;

together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also;
c) binders, e.g., magnesium aluminium silicate, starch paste, gelatin, tragacanth, methylcellulose, sodium carboxymethylcellulose and/or polyvinylpyrrolidone; if desired;
d) disintegrants, e.g., starches, agar, alginic acid or its sodium salt, or effervescent mixtures; and/or
e) absorbents, colourants, flavours and sweeteners.

Tablets may be either film coated or enteric coated according to methods known in the art.

Suitable compositions for oral administration include an effective amount of the active components described herein in the form of tablets, lozenges, aqueous or oily suspensions, dispersible powders or granules, food bar, confectionery, solution, emulsion, hard or soft capsules, a syrup, linctus, elixir, a liquid beverage or a foodstuff.

Compositions intended for oral use can be prepared according to any method known in the art for the manufacture of effective compositions; and such compositions can contain one or more additional agents selected from the group consisting of sweetening agents, flavouring agents, colouring agents and preserving agents in order to provide elegant and palatable preparations.

Tablets contain the composition comprising the active components herein described, in admixture with non-toxic orally acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets may be uncoated or coated by known techniques to delay disintegration and absorption in the gastrointestinal tract and thereby provide a sustained action over a longer period. For example, a time delay material such as glyceryl monostearate or glyceryl distearate can be employed.

Formulations for oral use can be presented as hard gelatin capsules wherein the composition comprising the active components is mixed with an inert solid diluent, for example, calcium carbonate, calcium phosphate or kaolin, or as soft gelatin capsules wherein the composition comprising the active components is mixed with water or an oil medium, for example, peanut oil, liquid paraffin or olive oil.

The soft capsule can be prepared using techniques well known in the art. For example, soft capsules are typically produced using a rotary die encapsulation process. Active agent formulations are fed into the encapsulation machine by gravity. In an embodiment, the formulation comprises pharmaceutical excipients such as olive oil, gelatin, glycerin, purified water, beeswax yellow, sunflower lecithin, silicon dioxide, titanium dioxide, F. D. & C Blue 1 and F. D. & C Red 4, microcrystalline cellulose, hypromellose, vegetable magnesium stearate, and/or silica.

A capsule shell can comprise one or more plasticizers such as glycerin, sorbitol, sorbitans, maltitol, glycerol, polyethylene glycol, polyalcohols with 3 to 6 carbon atoms, citric acid, citric acid esters, triethyl citrate and combinations thereof. In an embodiment, the plasticizer is glycerin.

In addition to the plasticizer(s), the capsule shell can include other suitable shell additives such as opacifiers, colourants, humectants, preservatives, flavourings, and buffering salts and acids.

Opacifiers are used to opacify the capsule shell when the encapsulated active agents are light sensitive. Suitable opacifiers include, but not limited to, titanium dioxide, zinc oxide, calcium carbonate and combinations thereof. In an embodiment, the opacifier is titanium dioxide.

Colourants can be used to for marketing and product identification and/or differentiation purposes. Suitable colourants include synthetic and natural dyes and combinations thereof.

Humectants can be used to suppress the water activity of the softgel. Suitable humectants include glycerin and sorbitol, which are often components of the plasticizer composition. Due to the low water activity of dried, properly stored softgels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

According to a further aspect of the invention there is provided a method of improving a cell's resistance to DNA damage, said method comprising the administration of an effective amount of a composition comprising an effective amount of one or more components, said components selected from acacetin, ACTI peptide, alpha-lipolic acid, alprostadil, anisomycin, apigenin, ascorbic acid, astragalus, berberine, β-lapachone, β-hydroxy-beta-methyl-butyrate, Bacopa monnieri, catechin, catechol, chamomile, chrysin, coumestrol, curcumin, dinitrophenol, dinoprost, ellagic acid, (−)-epigallocatechin gallate, green tea extract, fisetin, genistein, ginsenoside RE, glabridin, 18-α-glycyrrhetinic acid, 18-β-glycyrrhetinic acid, glycyrrhizin, hydroquinone, isoquercitrin (EMIQ), kaempferol, kuromanin, leucine, lithium, luteolin, luteolin, luteolinidin, melatonin, menadione, 1-methylnicotinamide (MNA), methyl salicylate, myricetin, nadide, niacin (vitamin B₃), nicotinamide (NAM), nicotinamide mononucleotide (NMN), nicotinamide riboside (NR), nicotinic acid adenine dinucleotide (NaAD), nicotinic acid mononucleotide (NaMN), parsley (Petroselinum crispum), phenylephrine, pokeweed mitogen, 15-Δ prostaglandin J2, puromycin, quercetin, quinolinic acid, retinoic acid, trichostatin A, troxrutin, rutin, tryptophan, vitamin D3, withaferin A, wortmannin and zinc (including salts thereof); and derivatives thereof; and any combination thereof.

According to a further aspect of the invention there is provided a method of improving a cell's DNA repair capacity, said method comprising the administration of an effective amount of a composition comprising an effective amount of one or more components, said components selected from acacetin, ACTI peptide, alpha-lipolic acid, alprostadil, anisomycin, apigenin, ascorbic acid, astragalus, berberine, β-lapachone, β-hydroxy-beta-methyl-butyrate, Bacopa monnieri, catechin, catechol, chamomile, chrysin, coumestrol, curcumin, dinitrophenol, dinoprost, ellagic acid, (−)-epigallocatechin gallate, green tea extract, fisetin, genistein, ginsenoside RE, glabridin, 18-α-glycyrrhetinic acid, 18-β-glycyrrhetinic acid, glycyrrhizin, hydroquinone, isoquercitrin (EMIQ), kaempferol, kuromanin, leucine, lithium, luteolin, luteolin, luteolinidin, melatonin, menadione, 1-methylnicotinamide (MNA), methyl salicylate, myricetin, nadide, niacin (vitamin B₃), nicotinamide (NAM), nicotinamide mononucleotide (NMN), nicotinamide riboside (NR), nicotinic acid adenine dinucleotide (NaAD), nicotinic acid mononucleotide (NaMN), parsley (Petroselinium crispum), phenylephrine, pokeweed mitogen, 15-4 prostaglandin J2, puromycin, quercetin, quinolinic acid, retinoic acid, trichostatin A, troxrutin, rutin, tryptophan, vitamin D3, withaferin A, wortmannin and zinc (including salts thereof); and derivatives thereof; and any combination thereof.

According to this aspect of the invention there is provided a method as herein described of enhancing cell resistance to DNA damage, oxidative stress, mitochondrial dysfunction, or improving DNA repair capacity.

The amount of the active components administered in the method according to this aspect of the invention may vary depending upon the nature of the active components, the mode of administration, etc. Exemplary amounts of active components which may be administered in the method are from about 1 mg to about 1000 mg; or from about 50 mg to about 900 mg; or from about 100 mg to about 800 mg; or from about 150 mg to about 700 mg; or from about 200 mg to about 600 mg; or from about 250 mg to about 500 mg. The aforementioned doses may be administered once daily, twice daily or up to three or four times a day.

According to a further aspect of the invention there is provided a method as herein described which comprise the mitigation, alleviation or improvement of the effects of ageing in a host.

According to this aspect of the invention there is provided a method of mitigation, alleviation or improvement of the effects of ageing in a host is by improving a cell's resistance to DNA damage and/or enhancing the cell's DNA repair capacity.

The method according to this aspect of the invention may comprise the mitigation, alleviation or improvement of age related skin conditions, skin conditions related to sun exposure, skin conditions related to pollution exposure, skin conditions related to oxidative stress, and skin conditions related to lifestyle choices, such as diet, alcohol and/or smoking. In addition, the method of the invention may be advantageous in the mitigation, alleviation or improvement of skin conditions related to inflammatory skin disorders and skin conditions related autoimmune disease skin disorders by improving a cell's resistance to DNA damage and/or enhancing the cell's DNA repair capacity.

In particular, according to this aspect of the invention there is provided a method of improving a skin cell's resistance to DNA damage and/or enhancing the skin cell's DNA repair capacity comprising topically applying an effective amount of the composition herein described to the skin.

Selection of a particular effective dose can be determined (e.g., via clinical trials) by a person skilled in the art based upon the consideration of several factors which will be known to the person skilled in the art, such as, the skin disorder to be mitigated, alleviated or improved; the nature and severity of the skin disorder being treated, the body mass of the host; and the like. The precise dose employed in the mitigation, alleviation or improvement of the skin disorder may also depend upon the route of administration. Effective doses can be extrapolated from dose-response curves derived from in vitro or animal model test systems.

However, in general, satisfactory results may be obtained at a daily dosage of the composition of the invention of from about 0.1 to about 500 mg/kg body weight; or from about 1 to about 400 mg/kg; or from about 1 to about 300 mg/kg; or from about 1 to about 200 mg/kg; or from about 1 to about 100 mg/kg; or from about 10 to about 50 mg/kg; administered, for example, in divided doses up to three or four times a day, e.g. twice daily, or in sustained release form.

It is often practical to administer the daily dose of the composition of the invention at various hours of the day. The amount of the active composition administered may depend on such factors as the solubility of the active composition, the formulation used, subject condition (such as weight), and/or the route of administration.

The present invention will now be described by way of example only and with reference to the accompanying figures, in which:

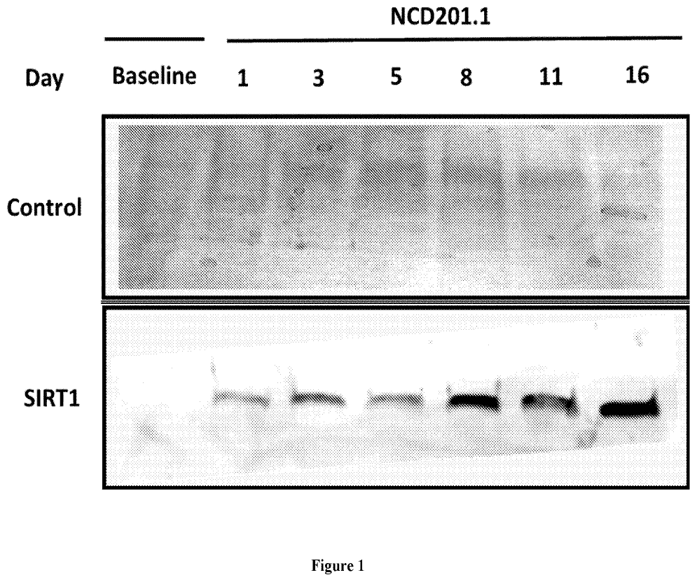

FIG. 1 illustrates potentiation of SIRT1 expression in human PBMCs from blood samples in a clinical trial over 16 days with exposure to the combination;

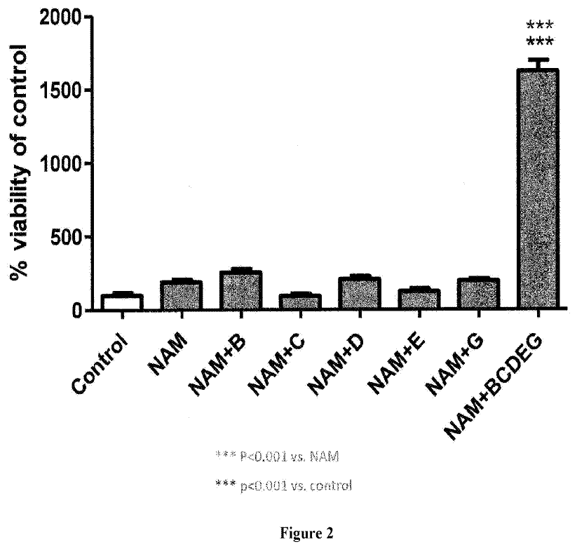

FIG. 2 illustrates HDF results for protection from oxidative stress; and

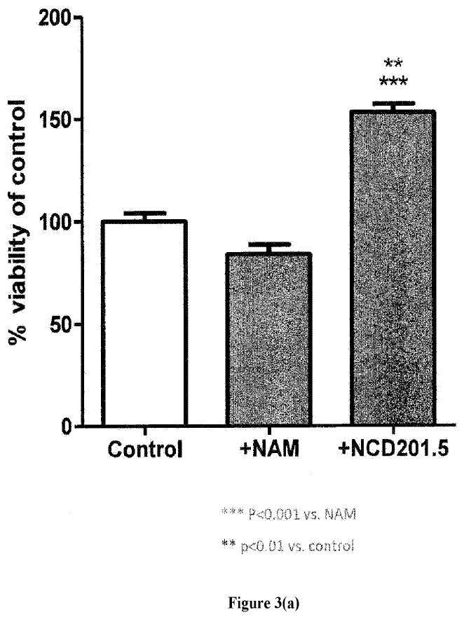

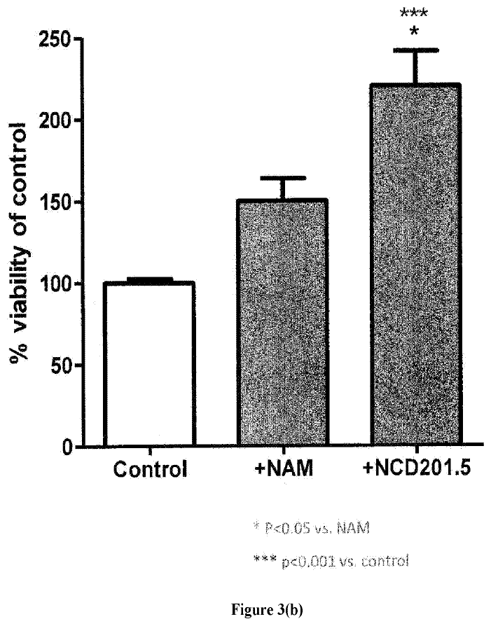

FIGS. 3(a) and (b) illustrate HDF results for protection from mitochondrial stress.

### EXPERIMENTAL

The measurement of DNA damage was performed using the ‘Fluorimetric Detection of Alkaline DNA Unwinding (FADU)’ method [Birnboim H C, Jevcak J J. Cancer Res (1981) 41:1889-1892]. This is a sensitive procedure to quantify DNA strand breaks and is based on the partial denaturation (“unwinding”) of double-stranded DNA under controlled alkaline conditions. Briefly, after infliction of DNA damage by irradiation, cell lysis was performed. Controlled unwinding of DNA was then performed under controlled conditions of pH and temperature. DNA strand breaks are sites susceptible to unwinding, thus, more DNA damage will result in more DNA unwinding. To terminate unwinding, a neutralising solution was added. To quantify the amount of DNA remaining double-stranded after the alkali incubation, a fluorescent probe was added that specifically binds to double stranded DNA. Low fluorescence intensities indicated a large number of DNA strand breaks present at the time of lysis. DNA repair was measured by allowing the cells to recover post irradiation (1 hour) and then measuring fluorescent intensity.

Human dermal fibroblasts were treated with our composition comprising a combination, which was niacinamide, quercetin, zinc citrate, ascorbic acid, apigenin and alpha-lipoic acid, for 24 hours prior to performing the FADU assay and DNA damage and repair levels were measured and compared to values from untreated (control) cells. Results were as follows:

| | |
|---|---|
| Damage | repair |

| | average | % cv | average | % cv |
|---|---|---|---|---|
| Composition | 0.00 | 8.51 | 100.00 | 8.91 |
| Control | 28.50 | 8.95 | 0.00 | 5.98 |

Values are average (and coefficient of variation as %) of at least 3 repeats for each condition. Results are in % and show:

$$DNA Damage ( \% ) = fluorescence signal intensity lost following 5 Gy irradiation$$

$$DNA Repair ( \% ) = \% of damage recovered after allowing for 1 h repair at 37 ° C .$$

Results show no loss in fluorescence intensity in cells treated with our composition compared to 28% loss in control cells, indicating that our composition protects the cells from DNA damage.

### Example 1

### SIRT1 Expression

Clinical participants' PBMCs (peripheral blood mononuclear cells) were analysed for changes in the expression of SIRT1 using Western blot analysis. SIRT1 expression increased markedly over a 16-day exposure to our composition. Increased SIRT1 expression indicates that beneficial downstream pathways associated with enhanced DNA repair were activated by our composition.

See FIG. 1.

### Clinical Results: Proteomics

Mass spectrometry was used to determine changes with the composition in levels of proteins associated with ageing.

Increased abundance of mitochondrial proteins was detected in volunteers' samples including:
Fumarate hydratase: Catalyses the hydration of fumarate to L-malate in the tricarboxylic acid (TCA) cycle to facilitate a transition step in the production of energy in the form of NADH.
Aspartate aminotransferase: Catalyses the transamination of the L-tryptophan metabolite L-kynurenine to form kynurenic acid (KA) as part of the De Novo pathway of NAD synthesis
VDAC2: A mitochondrial membrane channel
Complex IV subunits: Specifically, COX5A which is shown to decrease in skin with age, reducing mitochondrial function
Complex V subunits: Also found to decrease with age resulting in mitochondrial dysfunction.

Increased abundance of proteasome proteins were detected in volunteers' samples including:

19S and 20S proteasomal subunits: These subunits complex together to form the 26S proteasome which degrades damaged and unfolded proteins which are known to accumulate with age.

The proteasome is also important during mitochondrial biogenesis. This is because many mitochondrial proteins are synthesised in the cytoplasm and then need to be transported (unfolded) across the mitochondrial membrane. The proteasome ensures any damaged proteins are removed during this process. The 20S subunit is specifically implicated in the degradation of mitochondrial unfolded precursor proteins before their mitochondrial import, suggesting increased mitochondrial biogenesis with our composition.

Increased abundance of telomere-associated proteins was detected in volunteers' samples including:

RIF1: Telomere associated protein RIF-1 has been shown to be involved in the maintaining telomere length and in coordinating repair of telomere DNA damage.

We tested combination interventions in Human Dermal Fibroblasts (HDFs) against a variety of cellular stressors.

### Example 2

HDF Results: Protection from Oxidative Stress.
Pre-treatment with NAM or combinations
Followed by exposure to oxidative stress (tbh7ox)
Cell viability measured to assess protection from oxidative stress
Our composition provides much greater protection than niacinamide.
The composition is required to yield this enhanced protection, as predicted by systems pharmacology
The composition was alpha lipoic acid, apigenin, ascorbic acid, zinc citrate, niacinamide and quercetin.

The results are illustrated in FIG. 2.

### Example 3

HDF Results: Protection from Mitochondrial Stress

### Example 3(a)

Pre-treatment with NAM or our composition
Followed by exposure to mitochondrial stress (Rotanone—a Complex I inhibitor)
Cell viability measured to assess protection from mitochondrial dysfunction
Our composition provides much greater protection than niacinamide
The composition was alpha lipoic acid, apigenin, ascorbic acid, zinc citrate, and niacinamide.

The results are illustrated in FIG. 3(a).

### Example 3(b)

Pre-treatment with NAM or our composition
Followed by exposure to mitochondrial stress (NaN₃—a Complex IV inhibitor)
Cell viability measured to assess protection from mitochondrial dysfunction
Our composition provides much greater protection than niacinamide
The composition was alpha lipoic acid, apigenin, ascorbic acid, zinc citrate, and niacinamide.

The results are illustrated in FIG. 3(b).

1.-65. (canceled)

66. A composition comprising an effective amount of a combination of two or more components, said components selected from acacetin, ACTI peptide, alpha-lipolic acid, alprostadil, anisomycin, apigenin, ascorbic acid, astragalus, berberine, β-lapachone, β-hydroxy-beta-methyl-butyrate, Bacopa monnieri, catechin, catechol, chamomile, chrysin, coumestrol, curcumin, dinitrophenol, dinoprost, ellagic acid, (−)-epigallocatechin gallate, green tea extract, fisetin, genistein, ginsenoside RE, glabridin, 18-α-glycyrrhetinic acid, 18-β-glycyrrhetinic acid, glycyrrhizin, hydroquinone, isoquercitrin (EMIQ), kaempferol, kuromanin, leucine, lithium, luteolin, luteolin, luteolinidin, melatonin, menadione, 1-methylnicotinamide (MNA), methyl salicylate, myricetin, nadide, niacin (vitamin B₃), nicotinamide (NAM), nicotinamide mononucleotide (NMN), nicotinamide riboside (NR), nicotinic acid adenine dinucleotide (NaAD), nicotinic acid mononucleotide (NaMN), parsley (Petroselinium crispum), phenylephrine, pokeweed mitogen, 15-Δ prostaglandin J2, puromycin, quercetin, quinolinic acid, retinoic acid, trichostatin A, troxrutin, rutin, tryptophan, vitamin D3, withaferin A, wortmannin and zinc (including salts thereof); and derivatives thereof; and any combination thereof.

67. A method of enhancing cell resistance to DNA damage, oxidative stress, mitochondrial dysfunction, or improving DNA repair capacity, said method comprising the administration to an individual of an effective amount of a composition comprising an effective amount of from 150 mg to 700 mg niacinamide; from 150 mg to 700 mg quercetin or the derivative rutin; zinc (including salts thereof); ascorbic acid; from 1 mg to 1,000 mg parsley or the derivative apigenin; and from 150 mg to 700 mg alpha-lipoic acid.

68. A method according to claim 67 for use in enhancing cell resistance to DNA damage.

69. A method according to claim 67 for use in enhancing cell resistance to oxidative stress.

70. A method according to claim 67 for use in enhancing cell resistance to mitochondrial dysfunction.

71. A method according to claim 67 for use in improving a cell's DNA repair capacity.

72. A method according to claim 67 wherein the method comprises enhancing cell resistance to DNA damage, oxidative stress, mitochondrial dysfunction, and improving DNA repair capacity.

73. A method according to claim 67 wherein the method comprises the mitigation, alleviation or improvement of the effects of ageing in a host.

74. A method according to claim 73 wherein the method of mitigation, alleviation or improvement of the effects of ageing in a host is by improving a cell's resistance to DNA damage and/or enhancing the cell's DNA repair capacity.

75. A method according to claim 73 wherein the effects of ageing include age related skin conditions, skin conditions related to sun exposure, skin conditions related to pollution exposure, skin conditions related to oxidative stress, and skin conditions related to lifestyle choices, diet, alcohol and/or smoking.

76. A method according to claim 67 wherein the method comprises the mitigation, alleviation or improvement of inflammatory skin disorders and skin conditions related autoimmune disease skin disorders.

77. A method according to claim 76 wherein the skin condition is an age related skin condition.

78. A method according to claim 77 wherein the age related skin condition includes one or more of sagging, wrinkles, skin elasticity, skin ageing, skin moisture, wounds, acne, skin darkening, skin whitening, pigmentation, age-spots, loss of radiance, puffiness, uneven skin tone, redness, rosacea, loss of barrier function, loss of skin resilience, loss of firmness, stretch-marks, cellulite and dryness.

79. A method according to claim 77 wherein the skin condition is caused by sun exposure.

80. A method according to claim 77 wherein the skin condition includes one or more of actinic keratoses, freckles, lentigines or age spots, moles, photosensitivity, polymorphous light eruption, seborrheic keratoses, skin cancer (melanoma, squamous cell carcinoma, basal cell carcinoma), solar elastosis or wrinkles and sun burn.

81. A method according to claim 76 wherein the skin condition is caused by inflammation.

82. A method according to claim 76 wherein the skin condition includes one or more of acne, asteatotic eczema, atopic dermatitis, contact dermatitis, discoid eczema, eczematous drug eruptions, erythema multiforme, erythroderma, gravitational/varicose eczema, hand eczema, keratosis lichenoides chronica, lichen nitidus, lichen planus, lichen simplex, lichen striatus, mycosis fungoides, pityriasis lichenoides, psoriasis, seborrheic dermatitis, Stevens-Johnson Syndrome, toxic epidermal necrolysis and vasculitis.

83. A method according to claim 76 wherein the skin condition is caused by an autoimmune disease.

84. A method according to claim 67 for topical, transdermal, oral or parenteral administration.

85. A method according to claim 84 for topical administration.

86. A method according to claim 85 in the form of an aqueous solution, suspension, serum, ointment, cream, gel, sprayable formulation, transdermal patch or bandage.

87. A method according to claim 84 for parenteral administration.

88. A method according to claim 87 for parenteral administration in the form of an intramuscular, intravenous, subcutaneous, intraperitoneal, local or transdermal injections.

89. A method according to claim 84 for transdermal administration.

90. A method according to claim 89 in the form of an aqueous solution, suspension, ointment, cream, gel, sprayable formulation, transdermal patch or bandage.

91. A method according to claim 73 wherein the effects of ageing include increased frailty, loss of resilience, loss of muscle strength, loss of muscle endurance, loss of energy, loss of cognitive sharpness or loss of memory.

92. A method according to claim 73 wherein the effects of ageing include atherosclerosis, cardiovascular disease, cancer, arthritis, cataracts, osteoporosis, type 2 diabetes, hypertension or Alzheimer's disease.

93. A method according to claim 67 wherein the composition includes an effective amount of one or more glabridin, 18-α-glycyrrhetinic acid, 18-β-glycyrrhetinic acid and glycyrrhizin.
